(19) United States
(12) Patent Application Publication
(10) Pub. No.: US 2024/0288399 A1
(43) Pub. Date: Aug. 29, 2024

(54) METAL NANOPARTICLE-MAGNETIC PARTICLE COMPLEX, METHOD OF PREPARING SAME, AND USE THEREOF

(71) Applicant: KOREA ELECTRONICS TECHNOLOGY INSTITUTE, Seongnam-si (KR)

(72) Inventors: Seong Eun KIM, Seoul (KR); Se Eun JEONG, Seoul (KR); Seo Eun LEE, Seoul (KR)

(21) Appl. No.: 18/476,812

(22) Filed: Sep. 28, 2023

(30) Foreign Application Priority Data
Feb. 28, 2023 (KR) 10-2023-0027117

(51) Int. Cl. G01N 27/327; G01N 27/36; G01N 33/543

(52) U.S. Cl. CPC G01N 27/3278; G01N 27/3277; G01N 27/36; G01N 33/54326; G01N 33/5438; G01N 2446/20; G01N 2446/86

(57) ABSTRACT

Disclosed are a metal nanoparticle-magnetic particle complex, comprising a core comprising a magnetic particle, the first shell comprising metal nanoparticles and formed on the surface of the core, and the second shell comprising a response factor and formed on the surface of the first shell, a method of preparing the complex, and a method of measuring the concentration of a biomarker in a sample using the complex.

### BACKGROUND OF THE INVENTION

### Field of the Invention

The present disclosure relates to a metal nanoparticle-magnetic particle complex, a method of preparing the same, and the use thereof.

### Description of the Related Art

A biosensor is a signal conversion device configured to obtain useful information by detecting physical and chemical information from living organisms and measuring the same. As the medical paradigm in modern society shifts from treatment to precision medicine focused on prevention and diagnosis, the need for biosensors is rapidly increasing in medicine, and also biosensors are receiving attention in various fields such as the environment, food, and the like.

Today, as biosensors capable of on-site diagnosis, electrochemical sensors configured such that the concentration of a biomarker for a disease present in a sample collected from the human body is measured with an electrochemical signal are under study. In this disclosure, on-site diagnosis is diagnosis that is easily and quickly carried out by the patient or bystander on site (where the subject is located), unlike conventional diagnosis that was only possible in places equipped with specialized facilities such as hospitals, laboratories, etc.

Meanwhile, magnetic particles may be easily controlled by magnetism and have superior biocompatibility, so thorough research into use thereof in the biosensor field is ongoing.

### CITATION LIST

### Patent Literature

(Patent Document 1) KR 10-2022-0091033 A

### SUMMARY

### Technical Problem

The present disclosure is intended to provide a metal nanoparticle-magnetic particle complex, a method of preparing the same, and the use thereof.

### Technical Solution

The first aspect of the present disclosure provides a metal nanoparticle-magnetic particle complex, comprising a core comprising a magnetic particle, the first shell comprising metal nanoparticles and formed on the surface of the core, and the second shell comprising response factors and formed on the surface of the first shell.

In one embodiment, the diameter of the magnetic particle is 1 to 50 μm, and the diameter of the metal nanoparticles is 1 to 100 nm.

In one embodiment, the metal comprises gold, silver, nickel, platinum, aluminum, copper, an alloy of two or more thereof, or any combination thereof.

In one embodiment, the core further comprises the first linker compounds, and the first shell further comprises the second linker compounds that bind to the first linker compounds.

In one embodiment, the second shell further comprises the first linker compounds and the second linker compounds that bind to the first linker compounds.

In one embodiment, the first linker compound comprises avidin.

The second aspect of the present disclosure provides a method of preparing the metal nanoparticle-magnetic particle complex according to the first aspect, comprising (a) providing magnetic particles in a vessel, (b) adding metal nanoparticles to the vessel, and (c) adding response factors to the vessel.

In one embodiment, the magnetic particle is a magnetic particle conjugated with the first linker compounds, the metal nanoparticles are metal nanoparticles conjugated with the second linker compounds, and the first linker compounds bind to the second linker compounds.

In one embodiment, the method further comprises (d) adding the first linker compounds to the vessel after step (b) and before step (c).

In one embodiment, the response factor is a response factor conjugated with the second linker compound, and the first linker compound may bind to the second linker compound.

A third aspect of the present disclosure provides a method of measuring the concentration of a biomarker in a sample, comprising (A) immobilizing a plurality of metal nanoparticle-magnetic particle complexes on a working electrode, (B) bringing a sample into contact with the complexes, (C) bringing an electrical redox enzyme into contact with the complexes, and (D) measuring an electrical signal in response to electrical redox reaction from the working electrode, in which each of the complexes is the metal nanoparticle-magnetic particle complex according to the first aspect.

In one embodiment, step (A) further comprises (A-1) providing an electrode having the first surface and the second surface and having a magnet attached to at least a portion of the second surface and (A-2) immobilizing a plurality of metal nanoparticle-magnetic particle complexes on the first surface of the electrode.

In one embodiment, step (D) further comprises (D-1) immersing the working electrode in a solution including an electrical redox substrate and (D-2) applying a redox voltage to the working electrode.

### Advantageous Effects

The present disclosure provides a novel metal nanoparticle-magnetic particle complex, and provides a biosensor and biosensing methodology using the complex, with improved sensitivity and biocompatibility, and suitable for use on site.

### BRIEF DESCRIPTION OF THE DRAWINGS

FIG. 1 schematically shows a process of preparing a metal nanoparticle-magnetic particle complex and a biosensing process using the same according to one embodiment;

FIGS. 2 and 3 show results of measurement of electrical signals depending on the magnetic particle surface treatment conditions in an experimental example; and

FIGS. 4 and 5 show results of measurement of electrical signals depending on the type of purification solution and the concentration of disease-derived extracellular vesicles in an experimental example.

### DETAILED DESCRIPTION OF EMBODIMENTS

Aspects, specific advantages, and novel features of the present disclosure will become more apparent from the following detailed description and embodiments taken in conjunction with the accompanying drawings, but the present disclosure is not necessarily limited thereto. In addition, when describing the present disclosure, if it is determined that a detailed description of related known technology may unnecessarily obscure the gist of the present disclosure, the detailed description will be omitted.

### Complex

The first aspect of the present disclosure pertains to a metal nanoparticle-magnetic particle complex. The complex has a so-called “core-shell” structure in which an external shell surrounds an internal core. Particularly, the complex is configured to include a core, the first shell, and the second shell. The first shell may be formed on the surface of the core and configured to surround the surface of the core. The second shell may be formed on the outer surface of the first shell, namely a surface opposite to a surface of the first shell facing the surface of the core, and configured to surround the outer surface of the first shell.

The core of the present disclosure includes a magnetic particle. As used herein, the term “magnetic particle” refers to a magnetic bead that is able to stick to a magnet. Accordingly, the magnetic particle is not particularly limited, so long as it has the properties of a magnetic material and is able to be conjugated with the first linker compound described later. For example, ferrite such as magnetite (Fe₃O₄) or maghemite (γ-Fe₂O₃) may be used as the magnetic particle of the present disclosure. Here, ferrite has advantages such as superior magnetic properties, ease of size adjustment, and superior biocompatibility. In particular, magnetite is useful as the magnetic particle of the present disclosure because of low toxicity, strong supermagnetism, catalytic activity, and ease of preparation.

As will be described later, in the present disclosure, the magnetic particle may function as a biosupport that makes it possible to immobilize response factors on a working electrode of a biosensor. The use of the magnetic particle as a biosupport offers great advantages in immobilization and separation of a biosupport on and from an electrode substrate. Immobilization of the magnetic particle on an electrode substrate is possible simply using a magnet. Compared to conventional techniques that use materials other than the magnetic particle as a biosupport, separate treatment of the electrode substrate for immobilizing the biosupport is not required, which is desirable. Additionally, separation of the immobilized magnetic particle may be easily accomplished by removing the magnet from the electrode. This simple separation of the magnetic particle may also provide an advantage in that reuse of the working electrode used for sensing is possible after simple washing/drying. As used herein, the term “immobilization” includes both direct immobilization and indirect immobilization. Particularly, “immobilization” used herein includes not only direct immobilization of one object to another object without a separate medium, but also indirect immobilization in which two objects bind to each other through a separate medium.

In one embodiment of the present disclosure, the diameter of the magnetic particle is about 1 to 50 μm, about 1 to 30 μm, about 1 to 10 μm, or about 2 to 5 μm. If the diameter of the magnetic particle is less than the above lower limit, it may be difficult for metal nanoparticles to be immobilized on the surface of one magnetic particle, which may cause a problem of not forming a core-shell structure. On the other hand, if the diameter of the magnetic particle exceeds the above upper limit, a very large number of metal nanoparticles may be immobilized on the surface of one magnetic particle, which may cause excessive background noise or excessive nonspecific reaction when applied to a biosensor, undesirably lowering reliability in sensor accuracy.

The core may include the first linker compounds. As used herein, the term “linker compound” refers to a compound that indirectly connects one material to another material. The linker compound alone or in combination with another linker compound enables the connection. For example, biotin-avidin, which is a biochemical reaction conjugate, may be used as the linker compounds. Also, as a chemical covalent conjugate, a carbodiimide crosslinking agent or a succinimide crosslinking agent that binds to the primary amine of a protein may be used. From the viewpoint of controlling directionality of the connecting object, biotin-avidin may be used. Appropriate control of directionality of the connecting object may improve the surface area expression of the response factor, ultimately increasing the ability to detect a target material in the sample.

In one embodiment of the present disclosure, the magnetic particle of the core may be a magnetic particle conjugated with the first linker compound. Particularly, in order to form a core-shell structure, the magnetic particle may be a magnetic particle conjugated with first linker compounds, namely a magnetic particle coated with first linker compounds. The first linker compound may include avidin, biotin, or combinations thereof. The first linker compound may be avidin. Here, avidin may be neutravidin, natural avidin, streptavidin, CaptAvidin, or any combination thereof. One avidin may bind to four biotins. Therefore, in order for more metal nanoparticles to bind to one magnetic particle, the first linker compound may be avidin.

The first shell of the present disclosure includes metal nanoparticles. The first shell may include metal nanoparticles. When the magnetic particle is used as a biosupport, there is a disadvantage of high background noise compared to a conventional method of immobilizing a response factor on an ITO substrate through a linker compound. However, the presence of metal nanoparticles is capable of improving the transmission of electrical signals and biocompatibility and enabling binding of more enzymes through an enlarged surface area, thereby contributing to increasing sensitivity and accuracy of the biosensor. Accordingly, the metal of the present disclosure may be limited to a metal that may play the role described above, and the metal may be exemplified by gold, silver, nickel, platinum, aluminum, copper, an alloy of two or more thereof, or any combination thereof. The metal nanoparticles may be gold nanoparticles in view of sensitivity, accuracy, and superior biocompatibility of the biosensor.

In one embodiment of the present disclosure, the diameter of the metal nanoparticles may be about 1 to 100 nm, about 1 to 50 nm, about 10 to 40 nm, or about 10 to 30 nm. If the diameter of the metal nanoparticles is less than the above lower limit, the particle size may be excessively small, making it difficult to conjugate the linker compounds to the metal nanoparticles. On the other hand, if the diameter of the metal nanoparticles exceeds the above upper limit, a sufficient number of metal nanoparticles cannot be immobilized on the surface of one magnetic particle, making it difficult to expect improved efficiency due to formation of a metal nanoparticle-magnetic particle complex. The number of metal nanoparticles immobilized on the surface of one magnetic particle may be about 1000 to 5000, about 2000 to 5000, or about 3000 to 4000.

In one embodiment of the present disclosure, the first shell may include the second linker compounds. As used herein, the term “second linker compound” refers to a compound that may bind to the first linker compound described above. Accordingly, in the present disclosure, the second linker compound may vary depending on the first linker compound. For example, when the first linker compound is avidin, the second linker compound that binds thereto may be biotin. The second linker compound included in the first shell is a linker compound capable of binding to the first linker compound included in the core.

In one embodiment of the present disclosure, the metal nanoparticles of the first shell may be metal nanoparticles conjugated with the second linker compound. Particularly, the metal nanoparticles may be metal nanoparticles conjugated with second linker compounds, namely metal nanoparticles coated with second linker compounds. At least some of the second linker compounds applied onto the metal nanoparticles may bind to the first linker compounds of the core, and thus a structure in which the first shell surrounds the core may be formed.

The second shell includes a response factor. The second shell may include response factors. As used herein, the term “response factor” refers to a material that may react with and bind to a biomarker present in a sample (for example, when the biomarker is an antigen, the response factor is an antibody that is able to bind to the antigen). The response factor may vary depending on the type of biomarker, the presence or absence of which is to be confirmed in the sample. Accordingly, the response factor may be a predetermined response factor, and the complex of the present disclosure may be a complex custom-made for detection of a specific biomarker.

In one embodiment of the present disclosure, the second shell may include the first linker compound and the second linker compound. The second shell may include first linker compounds and second linker compounds. Some of the first linker compounds may bind to some of the second linker compounds of the first shell. Particularly, some of the first linker compounds of the second shell may bind to some of the second linker compounds of the first shell that do not bind to the first linker compound of the core.

The response factor of the second shell may be a response factor conjugated with the second linker compound. At least some of the second linker compounds of the second shell may bind to some of the first linker compounds of the second shell that do not bind to the second linker compound of the first shell. Accordingly, a structure in which the second shell surrounds the first shell may be formed.

Since both the first linker compound of the core and the first linker compound of the second shell have to be able to bind to the second linker compound of the first shell, the first linker compound of the core and the first linker compound of the second shell have to be compounds of the same kind. However, it is not necessary that the two first linker compounds be the same compound. For example, when the first linker compound of the core is streptavidin, the first linker compound of the second shell may be streptavidin or another avidin, but does not necessarily have to be streptavidin. Likewise, the second linker compound of the first shell and the second linker compound of the second shell may be compounds of the same kind, but they are not necessarily required to be the same compound.

The complex of the present disclosure described above may be prepared in advance to include a specific response factor that specifically reacts with a specific biomarker for the purpose of diagnosing a specific disease, and as described later, a plurality of complexes made of the complex of the present disclosure described above may be used for on-site biosensing.

### Method of Preparing Complex

The second aspect of the present disclosure pertains to a method of preparing a metal nanoparticle-magnetic particle complex. Unless otherwise stated, details about components corresponding to the first aspect may also be applied to the second aspect, and to avoid redundant description, details described in the first aspect may be omitted from the description of the second aspect.

The method of preparing the complex includes providing magnetic particles in a vessel. In the present disclosure, the vessel serves to perform adding, mixing, and binding of components of the complex, and the shape, size, etc. thereof are not particularly limited. For example, at a lab scale, examples of the vessel may include, but are not limited to, beakers, cylinders, flasks, bottles, etc. Also, at a plant scale, examples of the vessel may include, but are not limited to, reactors, etc.

In one embodiment of the present disclosure, the magnetic particle may be a magnetic particle conjugated with the first linker compound. Particularly, the magnetic particle may be a magnetic particle conjugated with first linker compounds. More particularly, the magnetic particle may be a magnetic particle coated with first linker compounds.

The preparation method includes adding metal nanoparticles to the vessel containing the magnetic particles. The metal nanoparticles may be added to the vessel and immobilized on the surface of the magnetic particles in the vessel. According to one embodiment of the present disclosure, the metal nanoparticles may be metal nanoparticles conjugated with the second linker compound. Particularly, the metal nanoparticles may be metal nanoparticles conjugated with second linker compounds. The second linker compound is a material that binds to the first linker compound described above. Accordingly, some of the first linker compounds conjugated to the magnetic particle and some of the second linker compounds conjugated to the metal nanoparticles may bind to each other, so that metal nanoparticles may be immobilized on the surface of a magnetic particle.

According to one embodiment of the present disclosure, the preparation method may further include incubating the mixture in the vessel after adding the metal nanoparticles to the vessel. The incubation may be performed at room temperature for a sufficient time to allow the metal nanoparticles to be immobilized on the surface of the magnetic particle. The incubation may be performed for about 30 minutes to 2 hours, about 30 minutes to 90 minutes, or about 30 minutes to 60 minutes. If the incubation time is less than the above lower limit, sufficient immobilization between the magnetic particle and the metal nanoparticles may not be achieved, which may result in a decrease in the final complex yield. As necessary, the incubation may further include mixing the mixture settled in the vessel.

The preparation method includes adding response factors to the vessel containing the metal nanoparticles. The response factors are immobilized on the surface of the metal nanoparticles. In one embodiment of the present disclosure, immobilization of the response factor may be achieved using a linker compound. As described above, when the metal nanoparticles are metal nanoparticles conjugated with the second linker compounds, the first linker compounds and the second linker compounds may be used to immobilize the response factors on the metal nanoparticles.

To this end, the preparation method may further include adding the first linker compounds to the vessel after adding the metal nanoparticles to the vessel and before adding the response factors to the vessel. The first linker compounds may bind to some of the second linker compounds conjugated to the metal nanoparticles that do not bind to some of the first linker compounds conjugated to the magnetic particle described above. Accordingly, the first linker compounds added to the vessel may be applied onto the surface of the metal nanoparticles in a form that surrounds the magnetic particle and the metal nanoparticles.

In order to ensure sufficient binding, the preparation method of the present disclosure may further include incubating the mixture in the vessel after adding the first linker compounds to the vessel. The incubation may be performed at room temperature for a sufficient time so that the first linker compounds added later are immobilized on the surface of the metal nanoparticles, particularly bind to the second linker compounds conjugated to the metal nanoparticles. The incubation may be performed for about 30 minutes to 2 hours, about 30 minutes to 90 minutes, or about 30 minutes to 60 minutes. If the incubation time is less than the above lower limit, sufficient immobilization between the second linker compounds and the metal nanoparticles may not be achieved, which may result in a decrease in the final complex yield.

In one embodiment of the present disclosure, the preparation method may further include, after adding the first linker compounds to the vessel and incubating the mixture in the vessel, adding BSA (bovine serum albumin) to the vessel and incubating the mixture in the vessel. The addition of BSA may contribute to reducing background noise and improving sensor reliability by preventing nonspecific binding of foreign materials and response factors described later to the magnetic particles and the metal nanoparticles.

As described above, when the linker compound is used to immobilize the response factor on the surface of the metal nanoparticles, the response factor may be a response factor conjugated with the second linker compound. When the first linker compounds are added to the vessel and then the response factors conjugated with the second linker compounds are added thereto, the first linker compounds and the second linker compounds may bind to each other. Accordingly, the response factor may be immobilized on the surface of the metal nanoparticles, ultimately preparing a complex as in the first aspect of the present disclosure including the magnetic particle as the core, the metal nanoparticles as the first shell, and the response factor as the second shell.

In order to ensure sufficient immobilization of the response factor on the surface of the metal nanoparticles, the preparation method of the present disclosure may further include incubating the mixture in the vessel after adding the response factors to the vessel. The incubation may be performed at room temperature for a sufficient time to allow the added response factors to be immobilized on the surface of the metal nanoparticles, particularly to allow the second linker compound conjugated to the response factor to bind to the first linker compound immobilized on the surface of the metal nanoparticles. The incubation may be performed for about 30 minutes to 2 hours, about 30 minutes to 90 minutes, or about 30 minutes to 60 minutes. If the incubation time is less than the above lower limit, sufficient immobilization between the response factor and the metal nanoparticles may not be achieved, which may result in a decrease in the final complex yield.

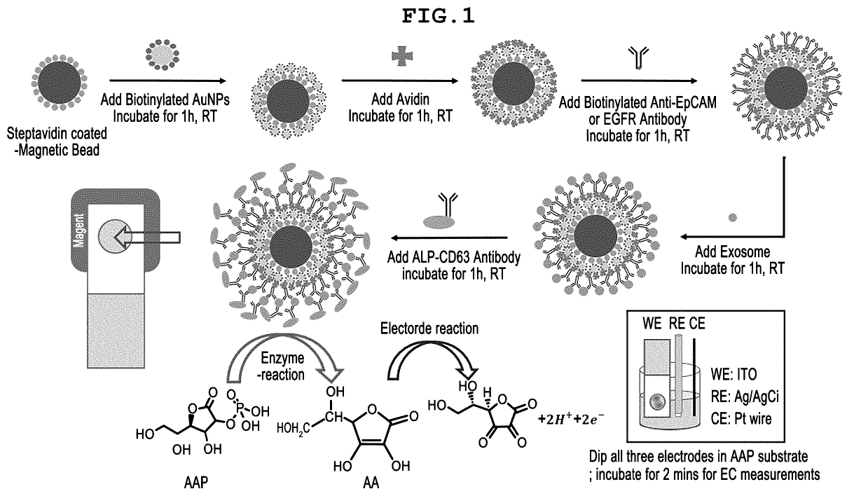

The method of preparing the complex according to one embodiment of the present disclosure is described below with reference to FIG. 1. FIG. 1 schematically shows a process of preparing a metal nanoparticle-magnetic particle complex and a biosensing process using the same according to one embodiment. The process of preparing the complex is shown in the top line of FIG. 1.

Referring to FIG. 1, a magnetic particle, the outer surface of which is coated with streptavidin, is provided in a vessel. Biotinylated gold nanoparticles are added to the vessel and incubated at room temperature for about 1 hour, so that the magnetic particle and the gold nanoparticles form a core-shell structure. Thereafter, avidin is added to the vessel and incubated at room temperature for about 1 hour. Thereafter, a biotinylated antibody is added to the vessel and incubated at room temperature for about 1 hour, thereby forming an additional shell structure by the antibody.

The metal nanoparticle-magnetic particle complex thus prepared may be applied to biosensing by direct contact with a sample containing a biomarker to be analyzed on site, as described later. As described above, the preparation method of the present disclosure is capable of immobilizing relatively large numbers of metal nanoparticles and response factors on one magnetic particle in a simple manner, and the presence of so many metal nanoparticles and response factors is expected to exhibit high sensitivity and biocompatibility in biosensing applications.

### Method of Measuring Concentration of Biomarker in Sample

A third aspect of the present disclosure pertains to a method of measuring the concentration of a biomarker in a sample using the complex described above. Unless otherwise stated, details about components corresponding to the first aspect and/or the second aspect may also be applied to the third aspect, and to avoid redundant description, details described in the first aspect and/or the second aspect may be omitted from the description of the second aspect.

The measurement method includes immobilizing a plurality of metal nanoparticle-magnetic particle complexes on a working electrode, bringing a sample into contact with the complexes, bringing an electrical redox enzyme into contact with the complexes, and measuring an electrical signal in response to electrical redox reaction from the working electrode.

The measurement method of the present disclosure includes immobilizing the complexes on the working electrode. Each of the complexes may be the complex according to the first aspect described above, particularly a complex prepared by the method according to the second aspect described above. In one embodiment of the present disclosure, magnetic particles and metal nanoparticles may be immobilized to each other, complexes to which response factors are not yet immobilized may be immobilized on a working electrode, and then the response factors may be immobilized on the complexes. Moreover, the immobilization may be performed before or after i) bringing the sample into contact with the complexes, and may even be performed after ii) bringing the electrical redox enzyme into contact with the complexes.

According to one embodiment of the present disclosure, immobilizing the complexes on the working electrode may further include providing an electrode having the first surface and the second surface and having a magnet attached to at least a portion of the second surface and immobilizing the complexes on the first surface of the electrode.

The working electrode of the present disclosure may be an ITO glass electrode or an Au electrode. From the viewpoint of minimizing background signals, an ITO glass electrode may be used. When applied to a biosensor, the ITO glass electrode is advantageous in that noise signals may be reduced and a substrate having an ITO thin film deposited on an organic substrate may be used as is without a separate adhesive layer. The ITO glass electrode may be advantageously manufactured as an electrode at very low cost because a process of manufacturing a thin film substrate having high and stable electrical conductivity may be equally utilized on a large substrate in the display industry.

The working electrode of the present disclosure may have the first surface and the second surface opposite the first surface and may have a magnet attached to at least a portion of the second surface. According to one embodiment, a magnet may be attached to the entire surface of the second surface. The means for attachment of the magnet is not particularly limited, so long as it does not prevent immobilization of magnetic particles on the first surface of the electrode by attractive force between the magnet and the magnetic particles.

A plurality of complexes may be immobilized on the first surface of the working electrode. Unlike conventional working electrodes on which response factors are immobilized through surface treatment with (3-aminopropyl)triethoxysilane (APTES) and glutaraldehyde (GA) after activation of OH groups on the surface of ITO glass, the working electrode of the present disclosure is characterized in that response factors are indirectly immobilized on the electrode surface by magnetic particles. Accordingly, it is possible to obviate the cumbersome surface pretreatment process required conventionally, thereby reducing required reagents and costs and also shortening the time necessary to perform the surface pretreatment process.

According to one embodiment of the present disclosure, in order to improve performance of the complexes, the complexes immobilized on the working electrode may be additionally subjected to stabilization and washing. In stabilization, the complexes may be stabilized by being attached to a magnet. The stabilization may be performed for about 5 to 25 minutes. Since the complexes are magnetic, the complexes may be uniformly distributed and aligned on the electrode surface by leaving the complexes on a magnet for a while. This enables stable immobilization of the complexes on the electrode surface. Moreover, in washing, foreign materials contained in the complexes (e.g., biomarkers that do not specifically bind to the complexes, electrical redox enzymes, materials other than the biomarkers in samples, etc.) may be removed from the complexes. The washing may be performed using, for example, distilled water. As necessary, stabilization and washing may be performed alternately at least twice. Performing the stabilization and washing as described above may contribute to high reliability in biosensing using the electrode.

The measurement method includes bringing the sample into contact with the complexes. In the present disclosure, the sample may be a bodily fluid obtained from an animal. More particularly, the sample may be a bodily fluid obtained from a human body. Examples of the bodily fluid may include, but are not limited to, blood, urine, saliva, and the like, and any bodily fluid may be used so long as the biomarker to be analyzed is present in the bodily fluid.

In the present disclosure, the biomarker may be an antigen, antibody, vitamin, protein, immune molecule, DNA, RNA, or the like. In one embodiment of the present disclosure, the biomarker may be present in exosomes or extracellular vesicles (EVs). The measurement method of the present disclosure may be applied on site. For example, it is possible to collect blood, particularly plasma, containing exosomes from a subject on site and use the same as a sample.

In order to ensure sufficient and specific binding between the response factor of the complex of the present disclosure and the biomarker in the sample, the measurement method of the present disclosure may further include performing incubation after bringing the sample into contact with the complexes. The incubation may be performed at room temperature (about 15-25° C.) for a sufficient time to allow the biomarker and the response factor to specifically bind to each other. The incubation may be performed for about 30 minutes to 2 hours, about 30 minutes to 90 minutes, or about 30 minutes to 60 minutes. If the incubation time is less than the above lower limit, sufficient binding between the response factor and the biomarker may not occur, making it difficult to accurately measure the concentration of the biomarker in the sample. On the other hand, if the incubation time exceeds the above upper limit, on-site application may become difficult due to the excessively long time.

The measurement method includes bringing the electrical redox enzyme into contact with the complexes. The biomarker described above may generally be a material incapable of oxidation or reduction reaction by itself. In order to determine the presence or absence of such a biomarker, the use of an electrical redox enzyme and substrate is considered.

In the present disclosure, the electrical redox enzyme is a material that activates the electrical redox substrate to enable redox reaction. Also, in the present disclosure, the electrical redox substrate is a material that, when a voltage is applied through the working electrode after being activated, is subjected to redox reaction by the applied voltage to absorb or release electrons, thereby generating a current.

Examples of the electrical redox enzyme may include ALP, HRP (horseradish peroxidase), glucose oxidase, luciferase, beta-D-galactosidase (β-malate dehydrogenase (MDH)), acetylcholinesterase, and the like. Most particularly, the enzyme may be ALP or HRP. ALP and HRP have the advantage of having very high reactivity, and in particular, HRP is advantageous in view of cost because it is inexpensive. Also, ALP is advantageous because of a long signal retention time of about 24 to 48 hours.

The enzyme may bind to a biomarker. In the present disclosure, the biomarker and the enzyme may bind to each other directly or indirectly. For indirect binding, for example, an enzyme conjugated with the secondary response factor may be used as the enzyme. Here, the secondary response factor is a response factor that is different from the primary response factor and is able to specifically bind to a biomarker (antigen).

In order to ensure sufficient and specific binding between the biomarker and the electrical redox enzyme, the measurement method of the present disclosure may further include incubating the mixture in the vessel after bringing the enzyme into contact with the complexes. The incubation may be performed at room temperature for a sufficient time to allow the biomarker and the enzyme to bind to each other. The incubation may be performed for about 30 minutes to 2 hours, about 30 minutes to 90 minutes, or about 30 minutes to 60 minutes.

The measurement method includes measuring the electrical signal in response to electrical redox reaction from the working electrode on which the complexes are immobilized. According to one embodiment of the present disclosure, measuring the electrical signal may further include immersing the working electrode on which the complexes are immobilized in a solution including an electrical redox substrate and applying a redox voltage to the working electrode.

The electrical redox substrate is determined depending on the electrical redox enzyme. For example, when ALP (alkaline phosphatase) is selected as the enzyme, ascorbic acid-2-phosphate (AAP) may be used as the substrate. AAP is converted into AA (ascorbic acid) as an enzyme reaction product due to separation of the phosphate functional group therefrom by ALP. AA is oxidized into dehydroascorbate by the applied voltage through the working electrode. An electrical signal generated during oxidation, for example, a current value, may be measured, and the concentration of the desired target material in the sample may be calculated therefrom.

In order to allow current to flow within the electrode system, other electrode(s) along with the working electrode may be immersed in the substrate solution during immersion. For a two-electrode system, a reference electrode along with the working electrode may be immersed in the substrate solution. For a three-electrode system, a reference electrode and a counter electrode along with the working electrode may be immersed in the substrate solution. A silver electrode and a platinum electrode may be used as the reference electrode and the counter electrode, respectively, but the present disclosure is not limited thereto.

Thereafter, an electrical signal in response to electrical redox reaction may be measured by applying a voltage to the electrodes. According to one embodiment, the measurement method may be performed by cyclic voltammetry (CV), chronoamperometry (CA), chronocoulometry (CC), or combinations thereof.

CV is a method of measuring current by circulating the voltage of the working electrode at a constant speed, through which a cyclic voltage-current curve may be obtained. Also, in CA and CC methods, when a sufficiently large voltage to induce electrochemical reaction is applied to a balanced electrode step by step, the flow of current is observed, and current and charge signals over time are observed with respect to the applied voltage step. Thereby, a time-to-current curve and a time-to-charge curve may be obtained. By comparing the curve thus obtained with a known database, it is possible to measure the concentration of the biomarker in the sample.

FIG. 1 shows the process of measuring the concentration of a biomarker according to one embodiment of the present disclosure. Referring again to FIG. 1, exosomes derived from a suspected disease subject are added to the vessel containing the complex prepared as described above and incubated at room temperature for about 1 hour. ALP conjugated with the second response factor that specifically binds to the biomarker is added to the vessel and incubated at room temperature for about 1 hour. Thereafter, the complex in which the biomarker and ALP are bound is recovered from the vessel, and the complex is immobilized on a surface of the working electrode opposite a surface with a magnet attached thereto, using attractive force between the magnet and the magnetic particle. Subsequently, the working electrode, a reference electrode, and a counter electrode are all immersed in an AAP substrate solution, after which the electrical signal is measured by applying a voltage.

Measuring the concentration of the biomarker in the sample as described above obviates conventional surface treatment required to immobilize the response factor on the working electrode surface and facilitates removal of the complex from the electrode surface after use, enabling reuse of the working electrode after simple washing. In addition, a biosupport used to allow the response factor to bind to the substrate is generally a non-conductor, and such a non-conductor has the problem of interfering with detection of electrochemical signals and adversely affecting detection sensitivity. However, the complex of the present disclosure includes a metal nanostructure with superior electrical conductivity, thereby reducing interference with the flow of electrochemical signals by the biosupport, and providing a high reaction specific surface area due to the spherical shape, making it possible to exhibit improved sensitivity and accuracy as a biosensor compared to conventional techniques.

A better understanding of the present disclosure may be obtained through the following examples. However, these examples are merely set forth to illustrate the present disclosure, and are not to be construed as limiting the present disclosure.

### EXAMPLES

### 1. Experiment of Measurement of Electrical Signal by Magnetic Particle Coating

### (1) Sample 1 (MB-AuNP-Avidin-ALP)

2 μl of streptavidin-coated magnetic particles (magnetic beads (MBs)) and 1 μl of biotin-conjugated gold nanoparticles (AuNPs) were placed in an e-tube and incubated at room temperature for about 1 hour. 15 μl of avidin was added to the e-tube and then incubated at room temperature for about 1 hour. Thereafter, 15 μl of biotinylated ALP was added thereto and incubated at room temperature for 1 hour, thereby preparing MB-AuNP-Avidin-ALP complexes.

The complexes thus prepared were removed from the e-tube, stabilized for about 10 minutes by being attached to a surface of an ITO glass electrode without a magnet opposite a surface with a magnet attached thereto, and washed with distilled water. The stabilization and washing were performed once more.

An electrode with the complexes immobilized thereon as a working electrode, an Ag/AgCl electrode as a reference electrode, and a Pt wire electrode as a counter electrode were used. All three electrodes were placed in a cell and immersed in 1 ml of AAP, and enzyme reaction was carried out for about 2 minutes. Thereafter, the oxidation peak for AA was detected by applying a voltage according to a CV method.

### (2) Sample 2 (MB-ALP)

MB-ALP complexes were prepared in the same manner as preparation of sample 1, with the exception that AuNPs and avidin were not added, after which the experiment was performed in the same manner as in sample 1.

### (3) Sample 3 (MB-AuNP-Avidin)

MB-AuNP-Avidin complexes were prepared in the same manner as preparation of sample 1, with the exception that ALP was not added, after which the experiment was performed in the same manner as in sample 1.

### (4) Sample 4 (MB)

MBs used in preparation of sample 1 were used alone, and the experiment was performed using the same in the same manner as in sample 1.

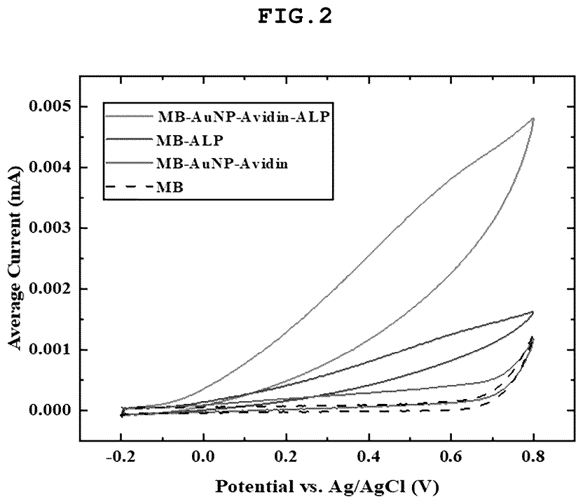

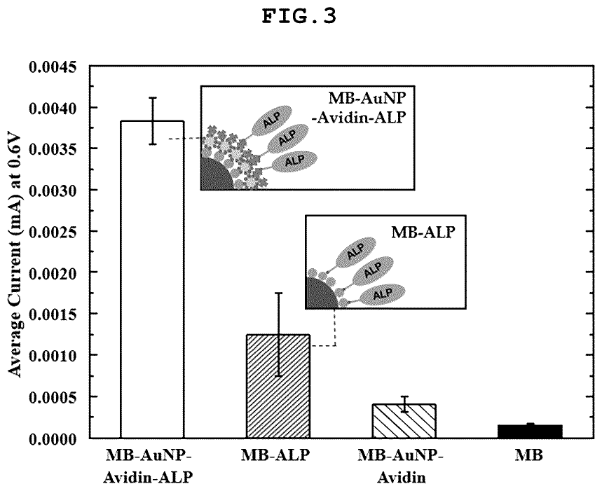

The experimental results of samples 1 to 4 are shown in FIGS. 2 and 3. FIG. 2 is a curve showing the average current at a voltage that changes according to a CV method. FIG. 3 is a graph comparing the average current values of samples at 0.6 V. The average current values at 0.6 V were about 3.82 E-03 mA in sample 1, about 1.25 E-03 mA in sample 2, about 4.04 E-04 mA in sample 3, and about 1.42 E-04 mA in sample 4.

From a comparison of samples 1 and 2, there was an increase in the average current value of about 206.40%, and from a comparison of samples 3 and 4, there was an increase in the average current value of about 178.61%. This difference is due to AuNPs present in the complexes of samples 1 and 3, and the presence of AuNPs is deemed to improve measurement sensitivity. Meanwhile, the current values of samples 3 and 4 in which ALP is not present may be considered to correspond to background noise of samples 1 and 2. Upon actual application, the results of samples 3 and 4 may be considered to correspond to the results that may be measured when no biomarker is present in the sample. By including AuNPs in the complexes, the average current value measured was increased regardless of the presence of ALP. This means that the addition of AuNPs may increase both measurement sensitivity of the sensor and background noise. However, as seen above, since the increase in current of sample 1 relative to sample 2 is greater than that of sample 3 relative to sample 4, the presence of metal nanoparticles in the complex of the present disclosure makes it possible to provide sensitivity to the biomarker greater than the increase in background noise, and ultimately, it is expected to exhibit superior performance compared to conventional sensors when used for a working electrode.

### 2. Experiment of Measurement of Electrical Signal by Sample Purification Solution

### (1) Preparation of Complex

15 μl of streptavidin-coated magnetic particles (magnetic beads (MBs)) and 1 μl of biotin-conjugated gold nanoparticles (AuNPs) were placed in an e-tube and incubated at room temperature for about 1 hour. After washing the mixture in the e-tube, the washing solution was removed, and then 15 μl of 1×PBS was added to the e-tube. Thereafter, 15 μl of the mixture in the e-tube was placed on a surface of an ITO glass electrode without a magnet opposite a surface with a magnet attached thereto, stabilized for about 10 minutes, and washed with distilled water. The stabilization and washing were performed once more. 15 μl of avidin was added to the mixture, followed by incubation at room temperature for about 1 hour. Thereafter, 15 μl of BSA (bovine serum albumin) was added to the mixture and incubated at room temperature for about 40 minutes. Thereafter, a biotinylated EGFR antibody was added to the e-tube and incubated at room temperature for about 1 hour, thereby preparing complexes with a core-shell structure of MB-AuNP-antibody.

### (2) Measurement of Electrical Signal

Purified glioblastoma-derived extracellular vesicles were added to the complexes prepared as described above, and incubated at room temperature for about 1 hour. Thereafter, ALP-conjugated anti-CD63 antibody was added to the complexes and incubated for 1 hour at room temperature.

An electrode with the complexes immobilized thereon as a working electrode, an Ag/AgCl electrode as a reference electrode, and a Pt wire electrode as a counter electrode were used. All three electrodes were placed in a cell and immersed in 1 ml of AAP, and enzyme reaction was carried out for about 2 minutes. Thereafter, the oxidation peak for AA was detected by applying a voltage according to a CC method.

The concentration of the vesicles and the type of purification solution for each sample are shown in Table 1 below. Here, the concentration of the vesicles is the amount of the vesicles present in the purification solution.

TABLE 1

| | Concentration of vesicles | | Type of purification solution |
|---|---|---|---|
| Sample A | 0 | ptcl/ml | PBS |
| Sample B | 10⁸ | ptcl/ml | PBS |
| Sample C | 0 | ptcl/ml | Human plasma |
| Sample D | 10⁸ | ptcl/ml | Human plasma |

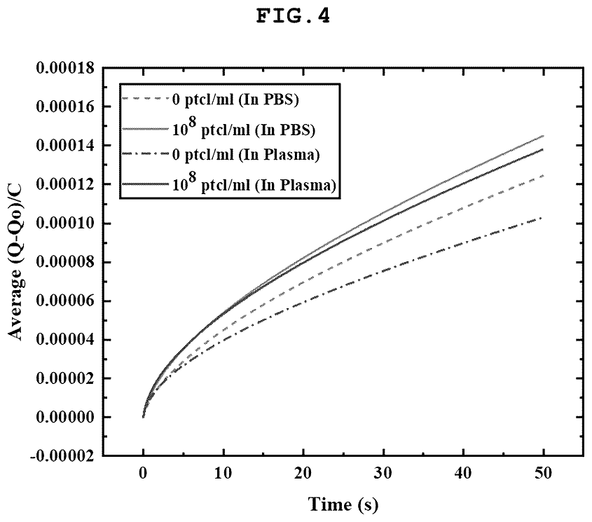

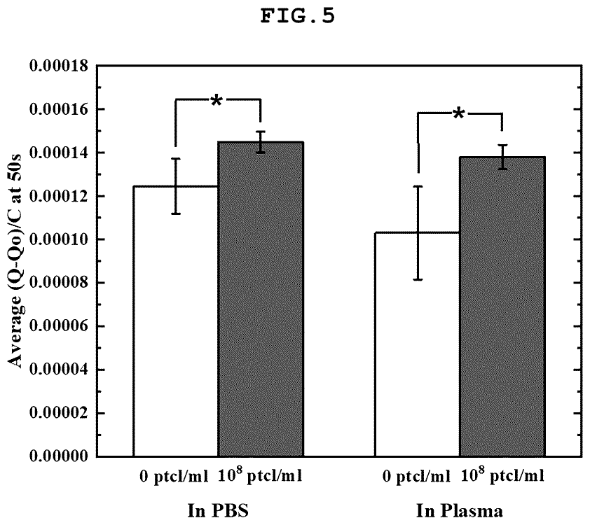

The experimental results of samples A to D are shown in FIGS. 4 and 5. FIG. 4 is a graph showing a change in average (Q-Q₀) value (unit: C) over time. FIG. 5 is a graph comparing the average (Q-Q₀) values of samples at 50 seconds. Here, Q represents the amount of charge and Q₀ represents the amount of charge at the start of the experiment. The signal values at 50 seconds were 1.24 E-04 C in sample A, about 1.45 E-04 C in sample B, about 1.03 E-04 C in sample C, and about 1.38 E-04 C in sample D. From a comparison of samples A and B, there was a signal increase of about 16.46%, and a p-value of about 0.02 was confirmed. Also, from a comparison of samples C and D, there was a signal increase of about 34.02%, and a p-value of about 0.05 was confirmed.

Based on the above experimental results, the difference in signal depending on the presence or absence of the biomarker was confirmed, and based on the results of samples B and D, it was confirmed that, when PBS was not used as the purification solution, particularly when plasma containing the relevant extracellular vesicles was directly collected from the subject and used as a sample, similar results to purifying extracellular vesicles with PBS were obtained. Thereby, the complex of the present disclosure can be found to be very useful from the viewpoint of on-site diagnosis of biomarker concentration.

Simple modifications or variations of the present disclosure fall within the scope of the present disclosure as defined in the accompanying claims.

1. A metal nanoparticle-magnetic particle complex, comprising:
a core comprising a magnetic particle;
the first shell comprising metal nanoparticles and formed on a surface of the core; and
the second shell comprising response factors and formed on a surface of the first shell.

2. The metal nanoparticle-magnetic particle complex according to claim 1, wherein a diameter of the magnetic particle is 1 to 50 μm, and a diameter of the metal nanoparticles is 1 to 100 nm.

3. The metal nanoparticle-magnetic particle complex according to claim 1, wherein the metal comprises gold, silver, nickel, platinum, aluminum, copper, an alloy of two or more thereof, or any combination thereof.

4. The metal nanoparticle-magnetic particle complex according to claim 1, wherein the core further comprises the first linker compounds, and the first shell further comprises the second linker compounds that bind to the first linker compound.

5. The metal nanoparticle-magnetic particle complex according to claim 1, wherein the second shell further comprises the first linker compounds and the second linker compounds that bind to the first linker compounds.

6. The metal nanoparticle-magnetic particle complex according to claim 4, wherein the first linker compound comprises avidin.

7. A method of preparing the metal nanoparticle-magnetic particle complex according to claim 1, comprising:
(a) providing magnetic particles in a vessel;
(b) adding metal nanoparticles to the vessel; and
(c) adding response factors to the vessel.

8. The method according to claim 7, wherein the magnetic particle is a magnetic particle conjugated with the first linker compounds, the metal nanoparticles are metal nanoparticles conjugated with the second linker compounds, and the first linker compounds bind to the second linker compounds.

9. The method according to claim 7, further comprising (d) adding the first linker compounds to the vessel after step (b) and before step (c).

10. The method according to claim 9, wherein the response factor is a response factor conjugated with the second linker compound, and the first linker compound binds to the second linker compound.

11. A method of measuring a concentration of a biomarker in a sample, comprising:
(A) immobilizing a plurality of metal nanoparticle-magnetic particle complexes on a working electrode;

1. A metal nanoparticle-magnetic particle complex, comprising:
a core comprising a magnetic particle;
the first shell comprising metal nanoparticles and formed on a surface of the core; and
the second shell comprising response factors and formed on a surface of the first shell.

2. The metal nanoparticle-magnetic particle complex according to claim 1,
wherein a diameter of the magnetic particle is 1 to 50 μm, and a diameter of the metal nanoparticles is 1 to 100 nm.

3. The metal nanoparticle-magnetic particle complex according to claim 1,
wherein the metal comprises gold, silver, nickel, platinum, aluminum, copper, an alloy of two or more thereof, or any combination thereof.

4. The metal nanoparticle-magnetic particle complex according to claim 1,
wherein the core further comprises the first linker compounds, and the first shell further comprises the second linker compounds that bind to the first linker compound.

5. The metal nanoparticle-magnetic particle complex according to claim 1, wherein the second shell further comprises the first linker compounds and the second linker compounds that bind to the first linker compounds.

6. The metal nanoparticle-magnetic particle complex according to claim 4, wherein the first linker compound comprises avidin.

7. A method of preparing the metal nanoparticle-magnetic particle complex according to claim 1, comprising:
(a) providing magnetic particles in a vessel;
(b) adding metal nanoparticles to the vessel; and
(c) adding response factors to the vessel.

8. The method according to claim 7, wherein the magnetic particle is a magnetic particle conjugated with the first linker compounds, the metal nanoparticles are metal nanoparticles conjugated with the second linker compounds, and the first linker compounds bind to the second linker compounds.

9. The method according to claim 7, further comprising (d) adding the first linker compounds to the vessel after step (b) and before step (c).

10. The method according to claim 9, wherein the response factor is a response factor conjugated with the second linker compound, and the first linker compound binds to the second linker compound.

11. A method of measuring a concentration of a biomarker in a sample, comprising:
(A) immobilizing a plurality of metal nanoparticle-magnetic particle complexes on a working electrode;
(B) bringing a sample into contact with the complexes;
(C) bringing an electrical redox enzyme into contact with the complexes; and
(D) measuring an electrical signal in response to an electrical redox reaction from the working electrode,
wherein each of the complexes is the metal nanoparticle-magnetic particle complex according to claim 1.

12. The method according to claim 11, wherein step (A) further comprises:
(A-1) providing an electrode having the first surface and the second surface and having a magnet attached to at least a portion of the second surface; and
(A-2) immobilizing a plurality of metal nanoparticle-magnetic particle complexes on the first surface of the electrode.

13. The method according to claim 11, wherein step (D) further comprises:
(D-1) immersing the working electrode in a solution comprising an electrical redox substrate; and
(D-2) applying a redox voltage to the working electrode.
